(19) United States
(12) Patent Application Publication
(10) Pub. No.: US 2008/0090865 A1
(43) Pub. Date: Apr. 17, 2008

(54) Antidiabetic Bicyclic Compounds

(76) Inventors: Min Ge, Edison, NJ (US); Lihu Yang, Edison, NJ (US); Changyou Zhou, Princeton, NJ (US); Songnian Lin, Monroe, NJ (US); Eric Dean Cline, Princeton, NJ (US)

Correspondence Address: MERCK AND CO., INC, P O BOX 2000, RAHWAY, NJ, 07065-0907, US

(21) Appl. No.: 11/794,591

(22) PCT Filed: Jan. 24, 2006
(86) PCT No.: PCT/US06/02395

§ 371 Date: Jun. 29, 2007

Related U.S. Application Data
(60) Provisional application No. 60/648,152, filed on Jan. 28, 2005.

(51) Int. Cl. A61K 31/47; A61K 31/435; A61P 3/10; C07D 215/00; C07D 221/02

(52) U.S. Cl. 514/312; 514/299; 546/153; 546/183

(57) ABSTRACT

Bicyclic compounds containing a fused pyridine ring, including pharmaceutically acceptable salts and prodrugs thereof, are agonists of G-protein coupled receptor 40 (GPR40) and are useful as therapeutic compounds, particularly in the treatment of Type 2 diabetes mellitus, and of conditions that are often associated with this disease, including obesity and lipid disorders, such as mixed or diabetic dyslipidemia, hyperlipidemia, hypercholesterolemia, and hypertriglyceridemia.

### FIELD OF THE INVENTION

The instant invention is concerned with bicyclic compounds containing a fused pyridine ring, including pharmaceutically acceptable salts and prodrugs thereof, which are agonists of G-protein coupled receptor 40 (GPR40) and are useful as therapeutic compounds, particularly in the treatment of Type 2 diabetes mellitus, and of conditions that are often associated with this disease, including obesity and lipid disorders.

### BACKGROUND OF THE INVENTION

Diabetes is a disease derived from multiple causative factors and characterized by elevated levels of plasma glucose (hyperglycemia) in the fasting state or after administration of glucose during an oral glucose tolerance test. There are two generally recognized forms of diabetes. In type 1 diabetes, or insulin-dependent diabetes mellitus (IDDM), patients produce little or no insulin, the hormone which regulates glucose utilization. In type 2 diabetes, or noninsulin-dependent diabetes mellitus (NIDDM), insulin is still produced in the body. Patients having type 2 diabetes have a resistance to the effects of insulin in stimulating glucose and lipid metabolism in the main insulin-sensitive tissues, which are muscle, liver and adipose tissues. These patients often have normal levels of insulin, and may have hyperinsulinemia (elevated plasma insulin levels), as they compensate for the reduced effectiveness of insulin by secreting increased amounts of insulin. Insulin resistance is not primarily caused by a diminished number of insulin receptors but rather by a post-insulin receptor binding defect that is not yet completely understood. This lack of responsiveness to insulin results in insufficient insulin-mediated activation of uptake, oxidation and storage of glucose in muscle, and inadequate insulin-mediated repression of lipolysis in adipose tissue and of glucose production and secretion in the liver.

Persistent or uncontrolled hyperglycemia that occurs with diabetes is associated with increased and premature morbidity and mortality. Often abnormal glucose homeostasis is associated both directly and indirectly with obesity, hypertension, and alterations of the lipid, lipoprotein and apolipoprotein metabolism, as well as other metabolic and hemodynamic disease. Patients with type 2 diabetes mellitus have a significantly increased risk of macrovascular and microvascular complications, including atherosclerosis, coronary heart disease, stroke, peripheral vascular disease, hypertension, nephropathy, neuropathy, and retinopathy. Therefore, therapeutic control of glucose homeostasis, lipid metabolism, obesity, and hypertension are critically important in the clinical management and treatment of diabetes mellitus.

Patients who have insulin resistance often have several symptoms that together are referred to as syndrome X, or the metabolic syndrome. According to one widely used definition, a patient having metabolic syndrome is characterized as having three or more symptoms selected from the following group of five symptoms: (1) abdominal obesity; (2) hypertriglyceridemia; (3) low high-density lipoprotein cholesterol (HDL); (4) high blood pressure; and (5) elevated fasting glucose, which may be in the range characteristic of Type 2 diabetes if the patient is also diabetic. Each of these symptoms is defined clinically in the Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with metabolic syndrome, whether or not they have or develop overt diabetes mellitus, have an increased risk of developing the macrovascular and microvascular complications that occur with type 2 diabetes, such as atherosclerosis and coronary heart disease.

There are several available treatments for type 2 diabetes, each of which has its own limitations and potential risks. Physical exercise and a reduction in dietary intake of calories often dramatically improve the diabetic condition and are the usual recommended first-line treatment of type 2 diabetes and of pre-diabetic conditions associated with insulin resistance. Compliance with this treatment is very poor because of well-entrenched sedentary lifestyles and excess food consumption, especially of foods containing high amounts of fat and carbohydrates. Pharmacologic treatments have focused on three areas of pathophysiology: (1) Hepatic glucose production (biguanides), (2) insulin resistance (PPAR agonists), and (3) insulin secretion.

The biguanides are a class of drugs that are widely used to treat type 2 diabetes. The two best known biguanides, phenformin and metformin, cause some correction of hyperglycemia. The biguanides act primarily by inhibiting hepatic glucose production, and they also are believed to modestly improve insulin sensitivity. The biguanides can be used as monotherapy or in combination with other anti-diabetic drugs, such as insulin or an insulin secretagogues, without increasing the risk of hypoglycemia. However, phenformin and metformin can induce lactic acidosis and nausea/diarrhea. Metformin has a lower risk of side effects than phenformin and is widely prescribed for the treatment of Type 2 diabetes.

The glitazones (i.e. 5-benzylthiazolidine-2,4-diones) are a newer class of compounds that can ameliorate hyperglycemia and other symptoms of type 2 diabetes. The glitazones that are currently marketed (rosiglitazone and pioglitazone) are agonists of the peroxisome proliferator activated receptor (PPAR) gamma subtype. The PPAR-gamma agonists substantially increase insulin sensitivity in muscle, liver and adipose tissue in several animal models of type 2 diabetes, resulting in partial or complete correction of elevated plasma glucose levels without the occurrence of hypoglycemia. PPAR-gamma agonism is believed to be responsible for the improved insulin sensititization that is observed in human patients who are treated with the glitazones. New PPAR agonists are currently being developed. Many of the newer PPAR compounds are agonists of one or more of the PPAR alpha, gamma and delta subtypes. Compounds that are agonists of both the PPAR alpha and PPAR gamma subtypes (PPAR alpha/gamma dual agonists) are promising because they reduce hyperglycemia and also improve lipid metabolism. The currently marketed PPAR gamma agonists are modestly effective in reducing plasma glucose and HemoglobinA1C. The currently marketed compounds do not greatly improve lipid metabolism and may actually have a negative effect on the lipid profile. Thus, the PPAR compounds represent an important advance in diabetic therapy, but further improvements are still needed.

Another widely used drug treatment involves the administration of insulin secretagogues, such as the sulfonylureas (e.g. tolbutamide and glipizide). These drugs increase the plasma level of insulin by stimulating the pancreatic β-cells to secrete more insulin. Insulin secretion in the pancreatic β-cell is under strict regulation by glucose and an array of metabolic, neural and hormonal signals. Glucose stimulates insulin production and secretion through its metabolism to generate ATP and other signaling molecules, whereas other extracellular signals act as potentiators or inhibitors of insulin secretion through GPCR's present on the plasma membrane. Sulfonylureas and related insulin secretagogues act by blocking the ATP-dependent K⁺ channel in β-cells, which causes depolarization of the cell and the opening of the voltage-dependent Ca2+ channels with stimulation of insulin release. This mechanism is non-glucose dependent, and hence insulin secretion can occur regardless of the ambient glucose levels. This can cause insulin secretion even if the glucose level is low, resulting in hypoglycemia, which can be fatal in severe cases. The administration of insulin secretagogues must therefore be carefully controlled. The insulin secretagogues are often used as a first-line drug treatment for Type 2 diabetes.

There has been a renewed focus on pancreatic islet-based insulin secretion that is controlled by glucose-dependent insulin secretion. This approach has the potential for stabilization and restoration of β-cell function. In this regard, several orphan G-protein coupled receptors (GPCR's) have recently been identified that are preferentially expressed in the β-cell and that are implicated in glucose stimulated insulin secretion (GSIS). GPR40 is a cell-surface GPCR that is highly expressed in human (and rodent) islets as well as in insulin-secreting cell lines. Several naturally-occurring medium to long-chain fatty acids (FA's) as well as synthetic compounds, including several members of the thiazolidinedione class of PPARγ agonists, have recently been identified as ligands for GPR40 (Itoh, Y. et al., Nature. 422: 173 [2003]; Briscoe, C. P. et al., J. Biol. Chem. 278: 11303 [2003]; Kotarsky, K. et al., Biochem. Biophys. Res. Comm. 301: 406 [2003]. Under hyperglycemic conditions, GPR40 agonists are capable of augmenting the release of insulin from islet cells. The specificity of this response is suggested by results showing that the inhibition of GPR40 activity by siRNA attenuates FA-induced amplification of GSIS. These findings indicate that, in addition to the intracellular generation of lipid-derivatives of FA's that are thought to promote insulin release, FA's (and other synthetic GPR40 agonists) may also act as extracellular ligands that bind to GPR40 in mediating FA-induced insulin secretion. There are several potential advantages of GPR40 as a potential target for the treatment of type 2 diabetes. First, since GPR40-mediated insulin secretion is glucose dependent, there is little or no risk of hypoglycemia. Second, the limited tissue distribution of GPR40 (mainly in islets) suggests that there would be less chance for side effects associated with GPR40 activity in other tissues. Third, GPR40 agonists that are active in the islets may have the potential to restore or preserve islet function. This would be highly advantageous, because long term diabetes therapy often leads to the gradual diminution of islet activity, so that after extended periods of treatment, it is often necessary to treat type 2 diabetic patients with daily insulin injections. By restoring or preserving islet function, GPR40 agonists may delay or prevent the diminution and loss of islet function in a type 2 diabetic patient.

### SUMMARY OF THE INVENTION

The class of compounds described herein is a new class of GPR40 agonists. The compounds are useful in the treatment of diseases that are modulated by GPR40 agonists, including type 2 diabetes, hyperglycemia that may be associated with type 2 diabetes or pre-diabetic insulin resistance, gestational diabetes, and obesity.

The present invention is directed to a compound of formula I, or a pharmaceutically acceptable salt thereof, including individual diastereomers and enantiomers or mixtures of diastereomers and/or enantiomers thereof, wherein:

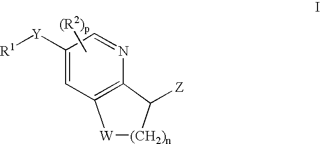

Z is selected from the group consisting of —CR³R⁴CO₂R⁵, —OCR³R⁴CO₂R⁵, —N(R⁶)(CR³R⁴CO₂R⁵), —SCR³R⁴CO₂R⁵, tetrazole, and the heterocyclic ring II:

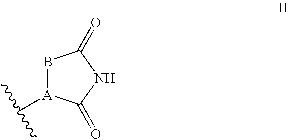

wherein A is —N— or —CR⁹—;

B is selected from S, —NR⁶—, —CH₂—, and O;

Y is selected from the group consisting of O, S, —C(═O)—, and —NR⁶—;

W is selected from O, S, —CH₂—, —CF₂—, and —NR⁶—;

R¹ is a cyclic substituent group selected from the group consisting of phenyl, naphthyl, C₃-C₆ cycloalkyl, indanyl, indenyl, tetrahydronaphthyl, 2,3-dihydrobenzofuranyl, benzopyranyl, 1,4-benzodioxanyl, pyridine, pyrazine, pyrimidine, furan, pyrrole, thiophene, imidazole, oxazole, thiazole, isoquinoline, isoxazole, isothiazole, pyrazole, oxadiazole, thiadiazole, triazole, tetrazole, triazine, thiene, pyridazine, pyrazine, benzisoxazole, benzoxazole, benzothiazole, benzimidazole, benzofurane, benzothiophene (including S-oxide and dioxide), furo(2,3-b)pyride, quinole, indole, isoquinole, quinazoline, and dibenzozofuran, wherein said R¹ is optionally substituted with 1-3 substituents independently selected from halogen, —OH, —CN, —NO₂, —NR⁷R⁸, C₁-C₃ alkyl, —OC₁-C₅ alkyl, —C(═O)C₁-C₃ alkyl, and —S(O)_{q}C₁-C₃ alkyl, wherein C₁-C₃ alkyl and the alkyl groups of —OC₁-C₅ alkyl, —C(═O)C₁-C₃ alkyl, and —S(O)_{q}C₁-C₃ alkyl are optionally substituted with 1-3 halogens;

R² is selected from the group consisting of halogen, —OH, —CN, —NO₂, —NR⁷R⁸, C₁-C₃ alkyl, and —OC₁-C₃ alkyl, wherein C₁-C₃ alkyl and the alkyl group of —OC₁-C₃ alkyl are optionally substituted with 1-3 halogens;

R³ and R⁴ are each independently selected from the group consisting of H and C₁-C₃ alkyl, which is optionally substituted with 1-3 F;

R⁵ is selected from the group consisting of H and C₁-C₆ alkyl, which is optionally substituted with 1-3 F;

R⁶, R⁷ and R⁸ are each independently selected from the group consisting of H and C₁-C₃ alkyl;

R⁹ is selected from the group consisting of H, C₁-C₃ alkyl, and CF₃;

n is an integer from 1-3;

p is 0, 1, or 2; and

q is 0, 1, or 2.

In the above definitions and subsequent definitions, alkyl groups may be either linear or branched, unless otherwise specified.

### DETAILED DESCRIPTION OF THE INVENTION

The invention has numerous embodiments, summarized below. These embodiments include the compounds, pharmaceutically acceptable salts of these compounds, and pharmaceutical compositions comprising these compounds and a pharmaceutically acceptable carrier. These embodiments may be especially useful in treating insulin resistance, type 2 diabetes, and dyslipidemia that is associated with type 2 diabetes and insulin resistance.

In a preferred subgroup of compounds having Formula I, R¹ is phenyl or 2-pyridinyl, wherein R¹ is optionally substituted with 1-3 substituents independently selected from halogen, —OH, —CN, —NO₂, —NR⁷R⁸, C₁-C₃ alkyl, —OC₁-C₅ alkyl, —C(═O)C₁-C₃ alkyl, and —S(O)_{q}C₁-C₃ alkyl, wherein C₁-C₃ alkyl and the alkyl groups of —OC₁-C₅ alkyl, —C(═O)C₁-C₃ alkyl, and —S(O)_{q}C₁-C₃ alkyl are optionally substituted with 1-3 halogens;

R³, R⁴, R⁵, and R⁶ are H;

R⁷ and R⁸ are independently selected from H and CH₃;

R⁹ is selected from H and C₁-C₃ alkyl; and

p is 0.

In many preferred compounds having Formula I, R⁷ and R⁸ are H; and R⁹ is selected from H and CH₃.

Many preferred subsets comprise compounds of Formula I in which R⁹ is H.

Many preferred subsets comprise compounds of Formula I in which R¹ is substituted with 1-3 groups independently selected from F, Cl, Br, CH₃, CF₃, —OCH₃, —OCF₃, —CN, —NO₂, and —OH.

In many preferred compounds of Formula I, Z is selected from —CH₂CO₂H and the heterocyclic ring IIa: 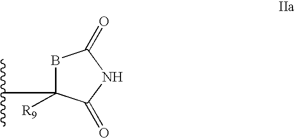
wherein R⁹ is selected from H and C₁-C₃ alkyl, and B is selected from S, O, and —NH—.

In many preferred compounds having Formula I, Y is O.

In many preferred compounds having Formula I, W is —CH₂—; and n is 1 or 2.

A preferred subset of compounds of Formula I has Formula Ia, 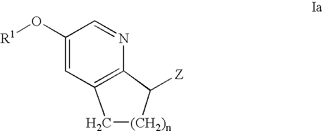
including pharmaceutically acceptable salts thereof and individual diastereomers and enantiomers or mixtures of diastereomers and/or enantiomers thereof, wherein:

Z is selected from the group consisting of —CH₂CO₂R⁵ and the heterocyclic ring IIa:

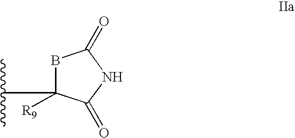

wherein B is selected from S, O, and —NH—;

R¹ is phenyl or 2-pyridinyl, wherein R¹ is optionally substituted with 1-3 substituents independently selected from F, Cl, Br, CH₃, CF₃, —OCH₃, —OCF₃, —CN, —NO₂, and —OH;

R⁵ is H or C₁-C₆ alkyl, which is optionally substituted with 1-3 F;

R⁹ is H or C₁-C₃ alkyl; and

n is 1 or 2.

In preferred compounds having Formula I or Ia, R⁵ and R⁹ are H.

In preferred compounds of Formula I and Formula Ia, B is S; and
R¹ is phenyl or 2-pyridinyl, where R¹ is substituted with 2 substituents independently selected from F, Cl, CH₃, and CF₃.

A highly preferred subgroup of compounds of Formula I has Formula Ib: 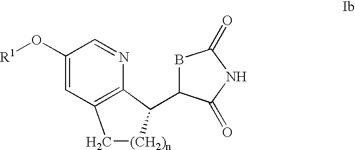
including pharmaceutically acceptable salts thereof, wherein:

R¹ is phenyl, 2-pyridinyl, indanyl, naphthyl, or quinolyl, which is optionally substituted with 1-3 substituents independently selected from F, Cl, Br, CH₃, CF₃, —OCH₃, —OCF₃, —CN, —NO₂, and —OH;

B is selected from S, O, and —NH—; and

n is 1 or 2.

The above compounds having formula Ib may be individual diastereomers or enantiomers or mixtures of diastereomers and/or enantiomers.

In a subgroup of Formula Ib, R¹ is phenyl or 2-pyridinyl, which is optionally substituted with 1-3 substituents independently selected from F, Cl, Br, CH₃, CF₃, —OCH₃, —OCF₃, —CN, —NO₂, and —OH.

In all of the compound and subsets of compounds described above and elsewhere herein, the “compounds” include pharmaceutically acceptable salts of the compounds, and when stereochemistry is not shown, include individual diastereomers or enantiomers, and all mixtures of diastereomers and/or enantiomers of the compounds.

Structures of specific compounds and synthetic methods for making the compounds are disclosed in the Examples. Structures and names of specific examples of the invention are disclosed in Table 1 below. Additional compounds are shown in Examples 8-13. The compounds in Table 1 and other examples also include pharmaceutically acceptable salts of the compounds, and when stereochemistry is not shown, individual diastereomers and enantiomers or mixtures of diastereomers and/or enantiomers of the compounds.

TABLE 1

| | |
|---|---|
| Ex. 1 | 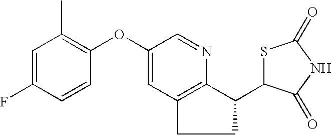 |
| | 5-[(R)-3-(4-Fluoro-2-methyl-phenoxy)-6,7-dihydro-5H-[1]pyrindin- |
| | 7-yl]-thiazolidine-2,4-dione |
| Ex. 2 | 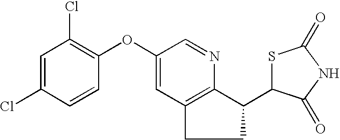 |
| | 5-[(R)-3-(2,4-Dichloro-phenoxy)-6,7-dihydro-5H-[1]pyrindin-7-yl]- |
| | thiazolidine-2,4-dione |
| Ex. 3 | 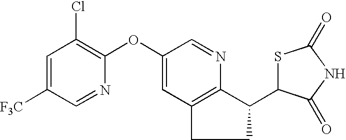 |
| | 5-[(R)-3-(3-Chloro-5-trifluoromethyl-pyridin-2-yloxy)-6,7-dihydro- |
| | 5H-[1]pyrindin-7-yl]-thiazolidine-2,4-dione |
| Ex. 4 | 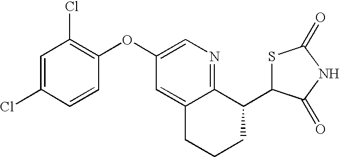 |
| | 5-[(R)-3-(2,4-Dichloro-phenoxy)-5,6,7,8-tetrahydro-quinolin-8-yl]- |
| | thiazolidine-2,4-dione |
| Ex. 5 | 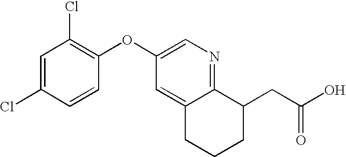 |
| | [3-(2,4-Dichloro-phenoxy)-5,6,7,8-tetrahydro-quinolin-8-yl]-acetic |
| | acid |
| Ex. 6 | 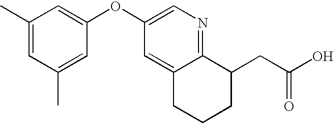 |
| | [3-(3,5-Dimethyl-phenoxy)-5,6,7,8-tetrahydro-quinolin-8-yl]-acetic |
| | acid |
| Ex. 7 | 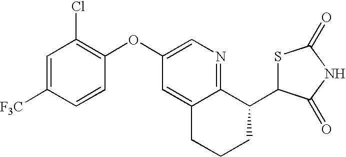 |
| | 5-[(R)-3-(2-Chloro-4-trifluoromethyl-phenoxy)-5,6,7,8-tetrahydro- |
| | quinolin-8-yl]-thiazolidine-2,4-dione |

The compounds of this invention may be used in pharmaceutical compositions comprising the compound or a pharmaceutically acceptable salt thereof and a pharmaceutically acceptable carrier. The compounds of this invention may be used in pharmaceutical compositions that include one or more other active pharmaceutical ingredients. The compounds of this invention may also be used in pharmaceutical compositions in which the compound of Formula I or a pharmaceutically acceptable salt thereof is the only active ingredient.

A compound of Formula I, or a pharmaceutically acceptable salt thereof, may be used in the manufacture of a medicament for the treatment of type 2 diabetes mellitus in a human or other mammalian patient.

A method of treating type 2 diabetes comprises the administration of a therapeutically effective amount of a compound of Formula I, or a pharmaceutically acceptable salt thereof, or a pharmaceutical composition comprising the compound, to a patient in need of treatment. Other medical uses of the compounds of Formula I are described hereinafter.

### DEFINITIONS

“Ac” is acetyl, which is CH₃C(═O)—.

“Alkyl” means saturated carbon chains which may be linear or branched or combinations thereof, unless the carbon chain is defined otherwise. Other groups having the prefix “alk”, such as alkoxy and alkanoyl, also may be linear or branched or combinations thereof, unless the carbon chain is defined otherwise. Examples of alkyl groups include methyl, ethyl, propyl, isopropyl, butyl, sec- and tert-butyl, pentyl, hexyl, heptyl, octyl, nonyl, and the like.

“Alkenyl” means carbon chains which contain at least one carbon-carbon double bond, and which may be linear or branched or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means a saturated carbocyclic ring, having a specified number of carbon atoms. The term may also be used to describe a carbocyclic ring fused to an aryl group. Examples of cycloalkyl include cyclopropyl, cyclopentyl, cyclohexyl, cycloheptyl, and the like. A “cycloalkenyl” ring is a cycloalkyl with one double bond.

“Aryl” (and “arylene”) when used to describe a substituent or group in a structure means a monocyclic, bicyclic or tricyclic compound in which all the rings are aromatic and which contains only carbon ring atoms (except as otherwise defined herein). “Heterocyclyl,” “heterocycle,” and “heterocyclic” means a fully or partially saturated monocyclic, bicyclic or tricyclic ring system containing at least one heteroatom selected from N, S and O, each of said rings having from 3 to 10 atoms, and may include a fused aryl ring. Examples of aryl substituents include phenyl and naphthyl. Aryl rings fused to cycloalkyls or cycloalkenyls are found in indanyl, indenyl, and tetrahydronaphthyl. Examples of aryl fused to heterocyclic groups are found in 2,3-dihydrobenzofuranyl, benzopyranyl, 1,4-benzodioxanyl, and the like. Examples of heterocycles include tetrahydrofuran, piperazine, piperidine, and morpholine. Preferred aryl groups are phenyl or naphthyl. Phenyl is generally the most preferred aryl group.

“Heteroaryl” (and heteroarylene) means a mono-, bi- or tricyclic aromatic ring containing at least one ring heteroatom selected from N, O and S (including SO and SO₂), with each ring containing 5 to 6 atoms, or as otherwise defined herein. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl (including S-oxide and dioxide), furo(2,3-b)pyridyl, quinolyl, indolyl, isoquinolyl, quinazolinyl, dibenzofuranyl, and the like.

“Halogen” includes fluorine, chlorine, bromine and iodine.

“Me” represents methyl.

The phrase “pharmaceutically acceptable” is employed herein to refer to those compounds, materials, compositions, salts and/or dosage forms which are, using sound medical judgment, and following all applicable government regulations, safe and suitable for administration to a human being or an animal.

The term “composition,” as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The substituent “tetrazole” means a 2H-tetrazol-5-yl substituent group and tautomers thereof.

Optical Isomers—Diastereomers—Geometric Isomers—Tautomers

Compounds of Formula I may contain one or more asymmetric centers and can thus occur as racemates, racemic mixtures, single enantiomers, diastereomeric mixtures and individual diastereomers. The present invention is meant to comprehend all such isomeric forms of the compounds of Formula I. Specifically, the compounds of the instant invention have at least one asymmetric center, which is on the ring that is fused to the pyridine ring at the point where the Z group is attached to the ring. There is also a second asymmetric center in the compounds that have a heterocyclic acid function, such as a thiazolidinedione or oxazolidinedione, at the point of attachment of the heterocyclic ring. Additional asymmetric centers may be present depending upon the nature of the various substituents on the molecule. Each such asymmetric center will independently produce two optical isomers, and it is intended that all of the possible optical isomers, stereoisomers, and diastereomers in mixtures and as pure or partially purified compounds are included within the scope of this invention (i.e. all possible combinations of the asymmetric centers as pure compounds or in mixtures).

Some of the compounds described herein may contain olefinic double bonds, and unless specified otherwise, are meant to include both E and Z geometric isomers.

Some of the compounds described herein may exist with different points of attachment of hydrogen, referred to as tautomers. An example is a ketone and its enol form, known as keto-enol tautomers. The individual tautomers as well as mixtures thereof are encompassed with compounds of Formula I.

Compounds of the Formula I having one or more asymmetric centers may be separated into diastereoisomers, enantiomers, and the like by methods well known in the art.

Alternatively, enantiomers and other compounds with chiral centers may be synthesized by stereospecific synthesis using optically pure starting materials and/or reagents of known configuration.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediainine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, or when it has a basic substituent group in its structure, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I are meant to also include the pharmaceutically acceptable salts.

Metabolites—Prodrugs

Therapeutically active metabolites, where the metabolites themselves fall within the scope of the claimed invention, are also compounds of the current invention. Prodrugs, which are compounds that are converted to the claimed compounds as they are being administered to a patient or after they have been administered to a patient, are also compounds of this invention.

Utilities

Compounds of the present invention are potent ligands for the GPR40 receptor and are agonists of the GPR40 receptor. The compounds of the invention, and pharmaceutically acceptable salts thereof, may be efficacious in the treatment of diseases that are modulated by GPR40 ligands and agonists. Many of these diseases are summarized below.

One or more of the following diseases may be treated by the administration of a therapeutically effective amount of a compound of this invention, or a pharmaceutically acceptable salt thereof, to a patient in need of treatment. Also, the compounds of the invention may be used for the manufacture of a medicament for treating one or more of these diseases:

(1) non-insulin dependent diabetes mellitus (type 2 diabetes);

(2) hyperglycemia;

(3) the metabolic syndrome;

(4) obesity;

(5) hypercholesterolemia;

(6) hypertriglyceridemia (elevated levels of triglyceride-rich-lipoproteins);

(7) mixed or diabetic dyslipidemia;

(8) low HDL cholesterol;

(9) high LDL cholesterol;

(10) hyperapoBliproteinemia; and

(11) atherosclerosis.

Preferred uses of the compounds are for the treatment of one or more of the following diseases by administering a therapeutically effective amount to a patient in need of treatment. The compounds may be used for manufacturing a medicament for the treatment of one or more of these diseases:

(1) Type 2 diabetes, and specifically hyperglycemia;

(2) Metabolic syndrome;

(3) Obesity; and

(4) Hypercholesterolemia.

The compounds are expected to be effective in lowering glucose, lipids, and insulin in diabetic patients and in non-diabetic patients who have impaired glucose tolerance and/or are in a pre-diabetic condition. The compounds may ameliorate hyperinsulinemia, which often occurs in diabetic or pre-diabetic patients, by modulating the swings in the level of serum glucose that often occurs in these patients. The compounds may also be effective in treating or reducing insulin resistance. The compounds may be effective in treating or preventing gestational diabetes.

The compounds, compositions, and medicaments as described herein may also be effective in reducing the risks of adverse sequelae associated with metabolic syndrome, and in reducing the risk of developing atherosclerosis, delaying the onset of atherosclerosis, and/or reducing the risk of sequelae of atherosclerosis. Sequelae of atherosclerosis include angina, claudication, heart attack, stroke, and others.

By keeping hyperglycemia under control, the compounds may also be effective in delaying or preventing vascular restenosis and diabetic retinopathy.

The compounds of this invention may have activity in improving or restoring β-cell function, so that they may be useful in treating type 1 diabetes or in delaying or preventing a patient with type 2 diabetes from needing insulin therapy.

The compounds generally may be efficacious in treating one or more of the following diseases: (1) type 2 diabetes (also known as non-insulin dependent diabetes mellitus, or NIDDM), (2) hyperglycemia, (3) low glucose tolerance, (4) insulin resistance, (5) obesity, (6) lipid disorders, (7) dyslipidemia, (8) hyperlipidemia, (9) hypertriglyceridemia, (10) hypercholesterolemia, (11) low HDL levels, (12) high LDL levels, (13) atherosclerosis and its sequelae, (14) vascular restenosis, (15) abdominal obesity, (16) retinopathy, (17) metabolic syndrome, (18) high blood pressure, and (19) insulin resistance.

One aspect of the invention provides a method for the treatment and control of mixed or diabetic dyslipidemia, hypercholesterolemia, atherosclerosis, low HDL levels, high LDL levels, hyperlipidemia, and/or hypertriglyceridemia, which comprises administering to a patient in need of such treatment a therapeutically effective amount of a compound having formula I. The compound may be used alone or advantageously may be administered with a cholesterol biosynthesis inhibitor, particularly an HMG-CoA reductase inhibitor such as lovastatin, simvastatin, rosuvastatin, pravastatin, fluvastatil, atorvastatin, rivastatin, itavastatin, or ZD-4522. The compound may also be used advantageously in combination with other lipid lowering drugs such as cholesterol absorption inhibitors (for example stanol esters, sterol glycosides such as tiqueside, and azetidinones such as ezetimibe), ACAT inhibitors (such as avasimibe), CETP inhibitors (for example torcetrapib), niacin, bile acid sequestrants, microsomal triglyceride transport inhibitors, and bile acid reuptake inhibitors. These combination treatments may be effective for the treatment or control of one or more related conditions selected from the group consisting of hypercholesterolemia, atherosclerosis, hyperlipidemia, hypertriglyceridemia, dyslipidemia, high LDL, and low HDL.

Administration and Dose Ranges

Any suitable route of administration may be employed for providing a mammal, especially a human, with an effective dose of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating or controlling diabetes mellitus and/or hyperglycemia or hypertriglyceridemia or other diseases for which compounds of Formula I are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams. In the case of a 70 kg adult human, the total daily dose will generally be from about 1 milligram to about 350 milligrams. For a particularly potent compound, the dosage for an adult human may be as low as 0.1 mg. The dosage regimen may be adjusted within this range or even outside of this range to provide the optimal therapeutic response.

Oral administration will usually be carried out using tablets or capsules. Examples of doses in tablets and capsules are 0.5 mg, 1 mg, 2 mg, 5 mg, 10 mg, 25 mg, 50 mg, 100 mg, 200 mg, and 350 mg. Other oral forms may also have the same or similar dosages.

Pharmaceutical Compositions

Another aspect of the present invention provides pharmaceutical compositions which comprise a compound of Formula I and a pharmaceutically acceptable carrier. The pharmaceutical compositions of the present invention comprise a compound of Formula I or a pharmaceutically acceptable salt as an active ingredient, as well as a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids. A pharmaceutical composition may also comprise a prodrug, or a pharmaceutically acceptable salt thereof, if a prodrug is administered.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds of Formula I can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds of formula I may also be administered parenterally. Solutions or suspensions of these active compounds can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Combination Therapy

Compounds of Formula I may be used in combination with other drugs that may also be useful in the treatment or amelioration of the diseases or conditions for which compounds of Formula I are useful. Such other drugs may be administered, by a route and in an amount commonly used therefor, contemporaneously or sequentially with a compound of Formula I. In the treatment of patients who have type 2 diabetes, insulin resistance, obesity, metabolic syndrome, and co-morbidities that accompany these diseases, more than one drug is commonly administered. The compounds of this invention may generally be administered to a patient who is already taking one or more other drugs for these conditions.

When a compound of Formula I is used contemporaneously with one or more other drugs, a pharmaceutical composition in unit dosage form containing such other drugs and the compound of Formula I is preferred. However, the combination therapy also includes therapies in which the compound of Formula I and one or more other drugs are administered on different overlapping schedules. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of Formula I.

Examples of other active ingredients that may be administered in combination with a compound of Formula I, and either administered separately or in the same pharmaceutical composition, include, but are not limited to:

(a) PPAR gamma agonists and partial agonists, including both glitazones and non-glitazones (e.g. troglitazone, pioglitazone, englitazone, MCC-555, rosiglitazone, balaglitazone, netoglitazone, T-131, LY-300512, and LY-818;

(b) biguanides such as metformin and phenformin;

(c) protein tyrosine phosphatase-1B (PTP-1B) inhibitors;

(d) dipeptidyl peptidase IV (DP-IV) inhibitors, such as MK-0431 and LAF-237;

(e) insulin or insulin mimetics;

(f) sulfonylureas such as tolbutamide and glipizide, or related materials;

(g) α-glucosidase inhibitors (such as acarbose);

(h) agents which improve a patient's lipid profile, such as (i) HMG-CoA reductase inhibitors (lovastatin, simvastatin, rosuvastatin, pravastatin, fluvastatin, atorvastatin, rivastatin, itavastatin, ZD-4522 and other statins), (ii) bile acid sequestrants (cholestyramine, colestipol, and dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) cholesterol absorption inhibitors, such as for example ezetimibe, (vi) acyl CoA:cholesterol acyltransferase (ACAT) inhibitors, such as avasimibe, (vii) CETP inhibitors, such as torcetrapib, and (viii) phenolic anti-oxidants, such as probucol;

(i) PPARα/γ dual agonists, such as muraglitazar, tesaglitazar, farglitazar, and JT-501;

(j) PPARδ agonists such as those disclosed in WO97/28149;

(k) antiobesity compounds such as fenfluramine, dexfenfluramine, phentiramine, subitramine, orlistat, neuropeptide Y5 inhibitors, Mc4r agonists, cannabinoid receptor 1 (CB-1) antagonists/inverse agonists, and P3 adrenergic receptor agonists;

(l) ileal bile acid transporter inhibitors;

(m) agents intended for use in inflammatory conditions such as aspirin, non-steroidal anti-inflammatory drugs, glucocorticoids, azulfidine, and cyclo-oxygenase 2 selective inhibitors;

(n) glucagon receptor antagonists;

(o) GLP-1,

(p) GIP-1,

(q) GLP-1 analogs, such as exendins, for example exenitide, and

(r) Hydroxysterol dehydrogenase-1 (HSD-1) inhibitors.

The above combinations include combinations of a compound of the present invention not only with one other active compound, but also with two or more other active compounds. Non-limiting examples include combinations of compounds having Formula I with two or more active compounds selected from biguanides, sulfonylureas, HMG-CoA reductase inhibitors, other PPAR agonists, PTP-1B inhibitors, DP-IV inhibitors, and anti-obesity compounds.

### Biological Assays

Generation of GPR40-Expressing Cells

Human and mouse GPR40 stable cell-lines were generated in CHO cells stably expressing NFAT BLA (Beta-lactamase). A human GPR40 stable cell-line was generated in HEK cells stably expressing the aequorin expressing reporter. The expression plasmids were transfected using lipofectamine (Life Technologies) following manufacturer's instructions. Stable cell-lines were generated following drug selection.

FLIPR Assays

FLIPR (Fluorimetric Imaging Plate Reader, Molecular Devices) assays were performed to measure agonist-induced calcium mobilization of the stable clones. For the FLIPR assay, one day before assay, GPR40/CHO NFAT BLA cells were seeded into black-wall-clear-bottom 384-well plates (Costar) at 1.4×10e4 cells/20 μl medium/well. The cells were incubated with 20 μl/well of the assay buffer (HBSS, 0.1% BSA, 20 mM HEPES, 2.5 mM probenecid, pH 7.4) containing 8 μM fluo-4,AM, 0.08% pluronic acid at room temperature for 100 minutes. Fluorescence output was measured using FLIPR. Compounds were dissolved in DMSO and diluted to desired concentrations with assay buffer. 13.3 μl/well of compound solution was added.

Inositol Phosphate Turnover Assay

The assay is performed in 96-well format. HEK cells stably expressing human GPR40 are plated to be 60-80% confluent within 72 hours. After 72 hours, the plates are aspirated and the cells washed with inositol-free DMEM (ICN). The wash media is replaced with 150 uL of 3H-inositol labeling media (inositol-free media containing 0.4% human albumin or 0.4% mouse albumin, 1× pen/strep antibiotics, glutamine, 25 mM HEPES to which is added 3H-myo-inositol NEN #NET114A 1 mCi/mL, 25 Ci/mmol diluted 1:150 in loading media with a final specific radioactivity of 1 uCi/150 uL). Alternatively, the human and mouse albumin can be added after the overnight labeling step before the addition of LiCl.

The assay is typically run the next day after 18 hours labeling. On the day of the assay, 5 uL of 300 mM LiCl is added to all wells and incubated at 37 degrees for 20 mins. 0.75 uL of 200× compounds are added and incubated with the cells for 60 minutes at 37 degrees. The media is then aspirated off and the assay terminated with the addition of 60 uL 10 mM formic acid. The cells are lysed for 60 mins at room temperature. 15-30 uL of lysate is mixed with 70 uL/1 mg YSi SPA beads (Amersham) in clear bottom Isoplates. The plates are shaken for 2 hours at room temperature. Beads are allowed to settle and the plates are counted in the Wallac Microbeta.

### In Vivo Studies

Male C₅₇BL/6N mice (7-12 weeks of age) are housed 10 per cage and given access to normal diet rodent chow and water ad libitum. Mice are randomly assigned to treatment groups and fasted 4 to 6 hours. Baseline blood glucose concentrations are determined by glucometer from tail nick blood. Animals are then treated orally with vehicle (0.25% methylcellulose) or test compound. Blood glucose concentration is measured at a set time point after treatment (t=0 min) and mice are then intraperitoneally-challenged with dextrose (2 g/kg). One group of vehicle-treated mice is challenged with saline as a negative control. Blood glucose levels are determined from tail bleeds taken at 20, 40, 60 minutes after dextrose challenge. The blood glucose excursion profile from t=0 to t=60 min is used to integrate an area under the curve (AUC) for each treatment. Percent inhibition values for each treatment are generated from the AUC data nonmalized to the saline-challenged controls.

### EXAMPLES

The following Examples are provided to illustrate the invention and are not to be construed as limiting the invention in any manner. The scope of the invention is defined by the appended claims.

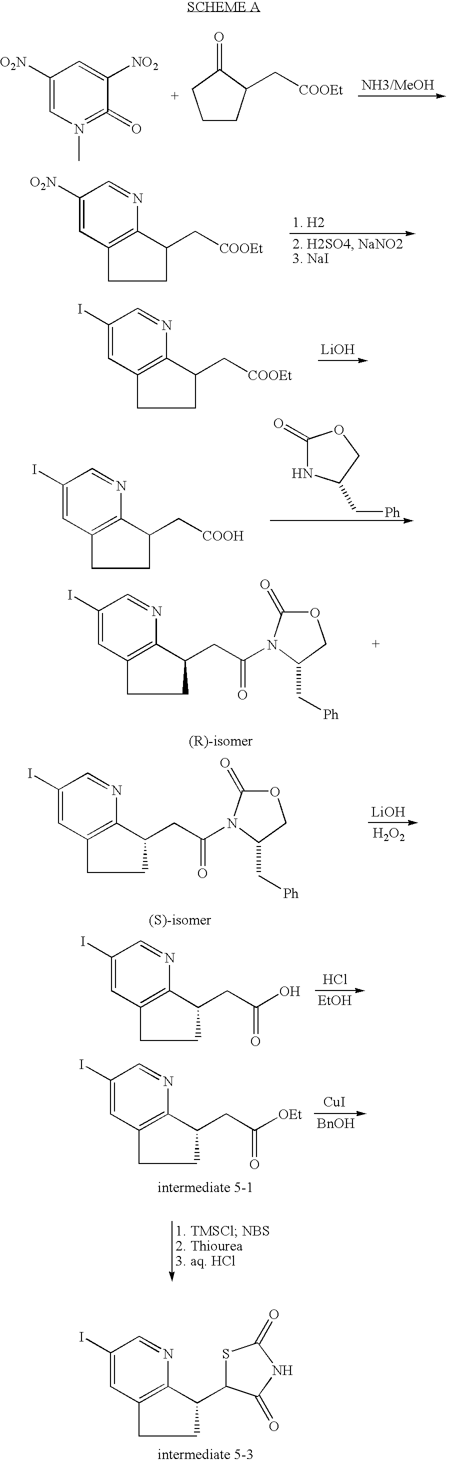

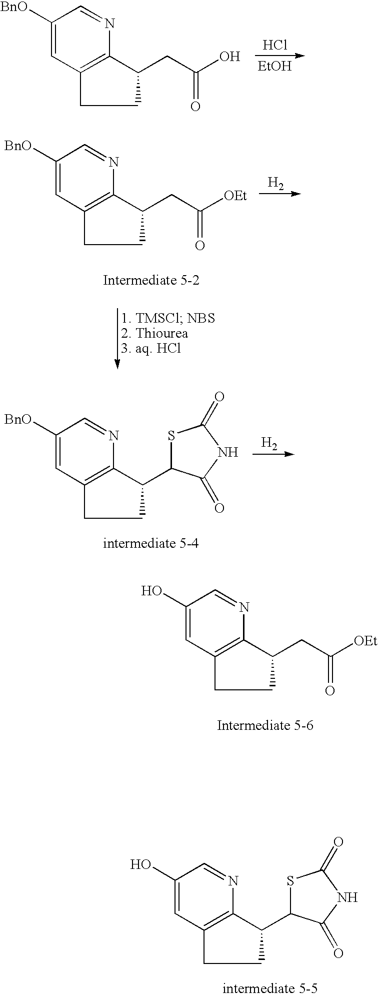

### Intermediate 5-1

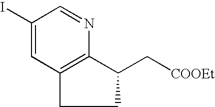 Step A

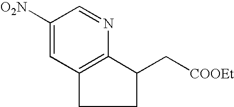

A mixture of 1-methyl-3,5-dinitro-2-pyridone (11.7 g, 59 mmol) and ethyl cyclopentanoneacetate (10 g, 59 mmol) in 2 M NH₃/MeOH (300 mL) was refluxed overnight and then cooled to room temperature. Methanol was removed in vacuo and the residue was partitioned between EtOAc (200 mL) and water (400 mL). The aqueous layer was further extracted with EtOAc (2×150 mL). The organic layers were combined, washed with brine (150 mL), dried over Na₂SO₄ and concentrated in vacuo. The residue was purified by flash chromatography (eluting with a gradient of 0 to 20% EtOAc/hexanes) to give pure product as light yellow oil. LC-MS for C₁₂H₁₅N₂O₄ [M+H⁺]: calculated 251.1, found 251.2.
Step B

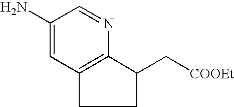

To a solution of the product from Step A (9.6 g, 38.4 mmol) in EtOH (200 mL) was added 10% Pd/C (5 g). The reaction was hydrogenated at 30 psi in a par-shaker for 1 hour. The mixture was then filtered through celite and the filtrate was concentrated to give pure product as yellow solid. LC-MS for C₁₂H₁₇N₂O₃ [M+H⁺]: calculated 221.1, found 221.2.
Step C

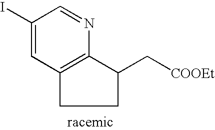

To a solution of product from Step B (2.9 g, 13.2 mmol) in 20% H₂SO₄ (30 mL) in an ice bath was added a solution of NaNO₂ (13.2 mmol, 911 mg) in water (5 mL) slowly. During the addition, the reaction temperature was carefully controlled under 4° C. by the addition of small amount of crushed ice. After addition, the reaction was further stirred at 0° C. for 30 minutes before a solution of NaI (15 mmol, 2.25 g) in water (10 mL) was added. After stirred for another 30 minutes, EtOAc (50 mL) was added and the reaction was carefully neutralized with solid NaHCO₃. The mixture was then partitioned between EtOAc (50 mL) and water (100 mL). The aqueous layer was further extracted with EtOAc (2×50 mL). The organic layers were combined, washed with brine (50 mL), dried over Na₂SO₄ and concentrated in vacuo. The residue was purified by flash chromatography (eluting with a gradient of 0 to 20% EtOAc/hexanes) to give pure product as light yellow oil. LC-MS for C₁₂H₁₅INO₂ [M+H⁺]: calculated 332.01, found 332.0.
Step D

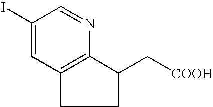

To a solution of product from Step C (17.5 mmol) in THF (60 mL), MeOH (40 mL) and water (10 mL) was added LiOH.H₂O (1.98 g). The reaction was stirred at room temperature overnight and then concentrated in vacuo. The residue was partitioned between water (400 mL) and CH₂Cl₂ (150 mL). The aqueous layer was separated ad acidified with 6 N HCl until pH=3 and then extracted with EtOAc (3×200 mL). The organic layers were combined, washed with brine (150 mL), dried over Na₂SO₄, filtered and concentrated in vacuo. The residue was purified by silica gel flash chromatography (eluting with 10% MeOH/CH₂Cl₂) to give pure product as light yellow solid. LC-MS for C₁₀H₁₁INO₂ [M+H⁺]: calculated 304.0, found 304.0.
Step E

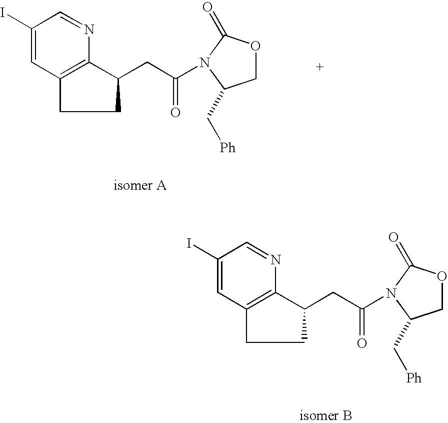

To a solution of product from Step D (4.6 g, 15.2 mmol) in THF (100 mL) was added Et3N (45.5 mmol, 6.4 mL) followed by PivCl (23 mmol, 2.8 mL) at 0° C. The reaction was allowed to warm up to room temperature over 30 minutes. Solid LiCl (24.3 mmol, 1.03 g) was then added to the reaction followed by (s)-4-benzyl-oxazolidione (22.8 mmol, 4.00 g). The mixture was further stirred at room temperate for 2 hours. The reaction was then partitioned between EtOAc (100 mL) and water (200 mL). The aqueous layer was further extracted with EtOAc (2×100 mL). The organic layers were combined, washed with brine, dried over Na₂SO₄, filtered and concentrated in vacuo. The residue was purified by silica gel flash chromatography (eluting with 4% EtOAc/CH₂Cl₂) to give isomer A (less polar isomer) and isomer B (more polar isomer), both as light yellow oil. LC-MS for C₂₀H₂₀IN₂O₃ [M+H⁺]: calculated 463.0, found 463.0.
Step F

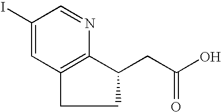

A solution of isomer B from Step E (2.9 g, 6.3 mmol) in THF (35 mL) and water (8 mL) was cooled to 0° C. in an ice bath. LiOH.H₂O (12.6 mmol, 530 mg) was added followed immediately by H₂O₂ (2.10 mL of 30% aqueous solution, 18.9 mmol). The reaction was monitored by TLC. After 4 hours at 0° C., the reaction was completed. Water (120 mL) was added and the mixture was washed with EtOAc (2×50 mL). The combined organic layers were extracted with water (1×50 mL). The aqueous layers were combined, acidified with 6N HCl (to pH=3) and then extracted with EtOAc (3×100 mL). The organic layers were combined, washed with brine, dried over Na₂SO₄, filtered and concentrated in vacuo. This crude compound was used in next step directly without further purification. LC-MS for C₁₀H₁₁INO₂ [M+H⁺]: calculated 304.0, found 304.0.
Step G

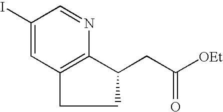

To a solution of crude product from Step F (˜6.3 mmol) in EtOH (50 mL) was added 4N HCl solution in dioxane (8 mL). The reaction was refluxed for 1.5 hr and then cooled down. The reaction was then concentrated in vacuo. The residue was partitioned between EtOAc (100 mL) and saturated NaHCO₃ aqueous solution (100 mL). The aqueous layer was further extracted with EtOAc (2×100 mL). The organic layers were combined, washed with Brine, dried over Na₂SO₄, filtered and concentrated in vacuo. The residue was purified by silica gel flash chromatography (eluting with 20% EtOAc/hexanes) to give Intermediate 5-1 as light yellow oil. LC-MS for C₁₂H₁₅INO₂ [M+H⁺]: calculated 332.01, found 332.0.

### Intermediate 5-2

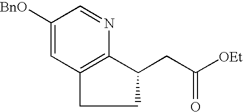 Step A

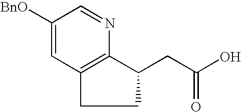

To a mixture of Intermediate 5-1 (315 mg, 0.952 mmol), 1,10-phenanthroline (0.19 mmol, 34 mg), Cs₂CO₃ (1.90 mmol, 620 mg) and CuI (0.0952 mmol, 19 mg) was added BnOH (1 mL). The reaction was heated at 120° C. for 4 hours and then cooled down. The reaction was then partitioned between EtOAc (20 mL) and water (50 mL). The aqueous layer was separated and further washed with EtOAc (2×20 mL). The organic layers were combined and extracted with water (1×20 mL). The aqueous layers were combined and acidified with 6 N HCl until pH=3 and then extracted with EtOAc (3×40 mL). The organic layers were combined, washed with brine, dried over Na₂SO₄, filtered and concentrated in vacuo to give crude product. LC-MS for C₁₇H₁₈NO₃ [M+H⁺]: calculated 284.1, found 284.1.
Step B

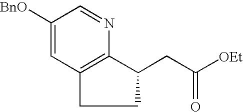

To a solution of crude product from Step A in EtOH (15 mL) was added 4N HCl in dioxane (3 mL). The reaction was refluxed for 2 hours, cooled down and then concentrated in vacuo. The residue was partitioned between EtOAc (30 mL) and saturated NaHCO₃ aqueous solution (50 mL). The aqueous layer was further extracted with EtOAc (2×30 mL). The organic layers were combined, washed with brine (30 mL), dried over Na₂SO₄, filtered and concentrated in vacuo. The residue was purified by silica gel flash chromatography (eluting with 20% EtOAc/hexanes) to give 148 mg (50% over 2 steps) of Intermediate 5-2 as light yellow oil. LC-MS for C₁₉H₂₂NO₃: calculated 312.1, found 312.1.

### Intermediate 5-3

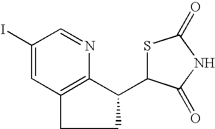 Step A

To a solution of NaHMDS (1.24 mmol, 1.24 mL of 1 M solution in THF) in THF (3 mL) was added a solution of Intermediate 5-1 (344 mg, 1.04 mmol) in THF (1 mL) at −78° C. After 30 minutes, TMSCl (1 M solution in THF, 1.24 mL) was added. After another 30 minutes at −78° C., NBS (1.2 mmol, 231 mg) was added in one portion. The reaction was allowed to warm up to 0° C. over 40 minutes and then quenched with aqueous NaHCO₃ solution (20 mL). The mixture was extracted with EtOAc (3×15 mL). The organic layers were combined, washed with brine (20 mL), dried over Na₂SO₄ and concentrated in vacuo. This material was used in next step directly. LC-MS for C₁₂H₁₄BrINO₂ [M+H⁺]: calculated 409.9, found 409.8.

Step B

To a suspension of crude product from Step A (1.04 mmol) in EtOH (5 mL) was added thiourea (1.4 mmol, 106 mg) and NaOAc (2 mmol, 164 mg). The reaction was heated at 85° C. overnight and then concentrated. To this residue was then added EtOH (6 mL) and 6 N HCl (3 mL). The reaction was heated at 85° C. overnight. The reaction was then concentrated in vacuo. The residue was purified on reverse phase HPLC (YMC-Pack Pro C18 5 micron, 20% to 80% CH₃CN/H₂O/0.1% TFA) to give Intermediate 5-3 as a solid. LC-MS for C₁₁H₁₀IN₂O₂S [M+H⁺]: calculated 360.99, found 361.0.

### Intermediate 5-4

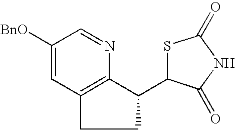

This was prepared from Intermediate 5-2 according to the procedure for Intermediate 5-3. LC-MS for C₁₈H₁₇N₂O₃S [M+H⁺]: calculated 341.1, found 341.1.

### Intermediate 5-5

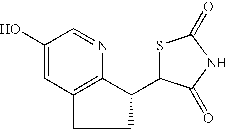

To a suspension of Intermediate 5-4 (300 mg, 0.88 mmol) in EtOH (20 mL) was added 4 N HCl in dioxane (500 μL) and 10% Pd/C (500 mg). The reaction was hydrogenated at 1 atm of H₂ for 2 hours to give a completed reaction. The mixture was then filtered through celite. The filtrate was concentrated in vacuo to give Intermediate 5-5 as a yellow solid. LC-MS for C₁₁H₁₁N₂O₃S [M+H⁺]: calculated 251.0, found 251.0.

### Intermediate 5-6

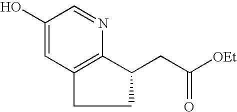

This was prepared from Intermediate 5-2 according to the procedure for Intermediate 5-5. LC-MS for C₁₂H₁₆NO₃ [M+H⁺]: calculated 222.1, found 222.1.

### Example 1

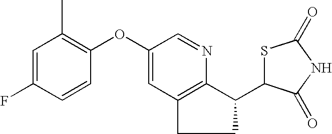

To a mixture of Intermediate 5-3 (0.1 mmol, 36 mg), CuI (0.02 mmol, 4 mg), N,N-dimethyl glycine HCl salt (0.06 mmol, 8.4 mg), 2-methyl-4-fluoro-phenol (0.15 mmol, 19 mg) and Cs₂CO₃ (0.47 mmol, 153 mg) was added dioxane (1 mL). The reaction was heated at 95° C. overnight and then filtered. The filtrate was acidified with TFA (0.5 mL) and then concentrated. The residue was purified by reverse phase HPLC (YMC-Pack Pro C18 5 micron, 20% to 80% CH₃CN/H₂O/0.1% TFA) to give pure product as a solid. LC-MS for C₁₈H₁₆FN₂O₃S [M+H⁺]: calculated 359.1, found 359.0.

### Example 2

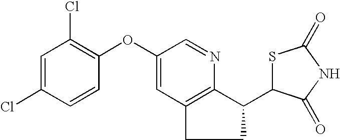

To a mixture of Intermediate 5-3 (0.1 mmol, 36 mg), CuI (0.02 mmol, 4 mg), N,N-dimethyl glycine HCl salt (0.06 mmol, 8.4 mg), 2,4-dichloro-phenol (0.15 mmol, 25 mg) and Cs₂CO₃ (0.47 mmol, 153 mg) was added dioxane (1 mL). The reaction was heated at 95° C. overnight and then filtered. The filtrate was acidified with TFA (0.5 mL) and then concentrated. The residue was purified by reverse phase HPLC (YMC-Pack Pro C18 5 micron, 20% to 80% CH₃CN/H₂O/0.1% TFA) to give pure product as a solid. LC-MS for C₁₇H₁₃Cl₂N₂O₃S [M+H⁺]: calculated 395.0, found 394.9.

### Example 3

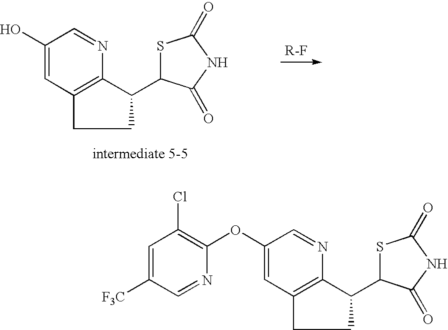

To a solution of Intermediate 5-5 (25 mg, 0.1 mmol) in DMF (0.5 mL) was added Cs₂CO₃ (0.3 mmol, 98 mg) followed by 3-chloro-2-fluoro-5-(trifluoromethyl)pyridine (0.15 mmol). The reaction was heated at 50° C. for 1 hr, diluted with CH₃CN (1 mL) and then acidified with trifluoroacetic acid (0.4 mL). The mixture was purified by reverse phase HPLC (YMC-Pack Pro C18 5 micron, 20% to 80% CH₃CN/H₂O/0.1% TFA) to give pure product as a solid. LC-MS for C17H₁₂ClF₃N₃O₃S [M+H⁺]: calculated 430.0, found 430.0.

### Intermediate 6-1

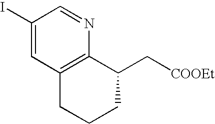

This was prepared from ethyl cyclohexanoneacetate according to the procedure for Intermediate 5-1. LC-MS for C₁₃H₁₇INO₂ [M+H⁺]: calculated 346.0, found 346.0.

### Intermediate 6-2

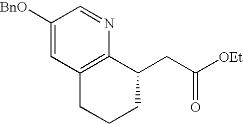

This is prepared from Intermediate 6-1 using the procedure that is used to convert Intermediate 5-1 to 5-2.

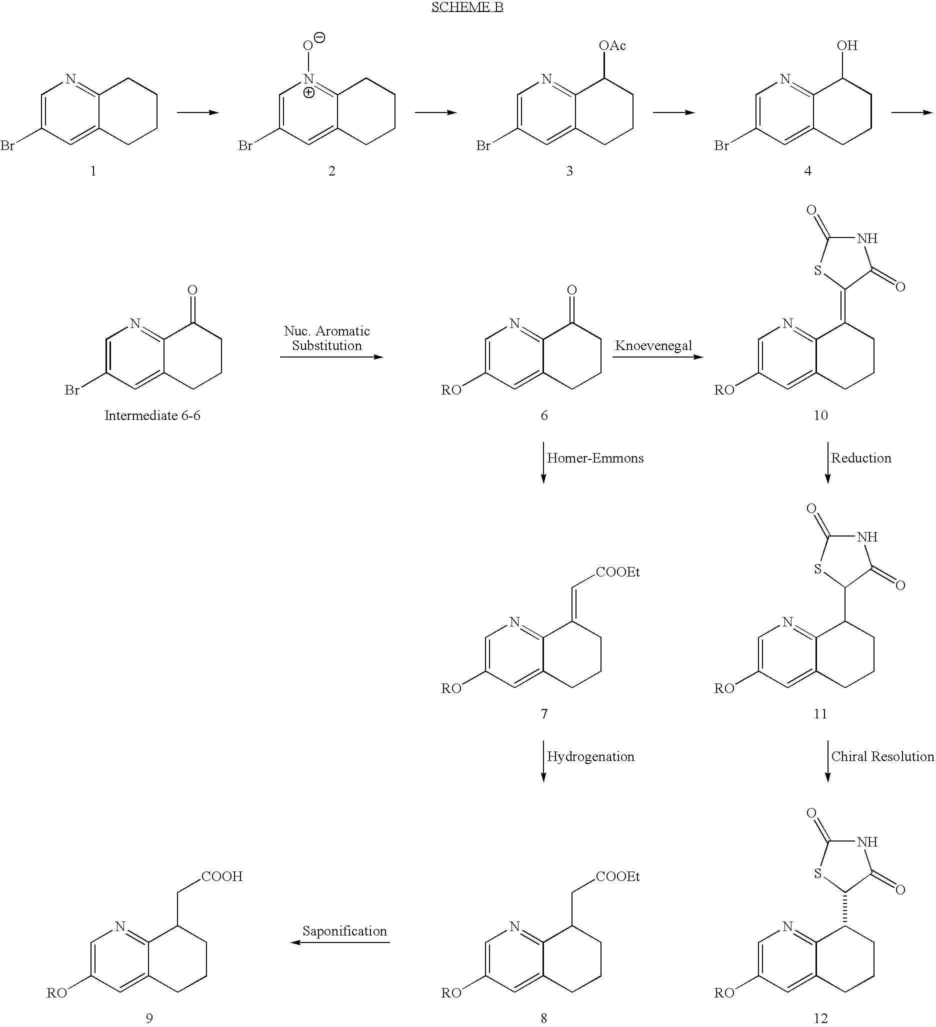

### Intermediate 6-6

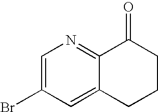 Step A

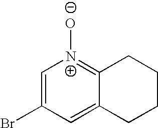

To a solution of 3-bromo-5,6,7,8-tetrahydroquinoline (15 g, 70 mmol) in dichloromethane (200 mL) was added 3-chloroperoxybenzoic acid (77% max, 31.7 g, 140 mmol). The reaction was stirred at reflux for 2 hours. To the cooled reaction mixture was added calcium hydroxide (21 g, 280 mmol) and the solution was stirred overnight. The solid was removed by vacuum filtration and washed with dichloromethane (100 mL). The combined filtrate and wash was concentrated in vacuo. The crude product was taken directly to the next reaction. LC-MS for C₉H₁₀BrNO [M+H⁺]: calculated 228.0, found 228.0.
Step B

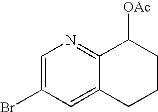

To the crude product from Step A (approx 70 mmol) was added acetic anhydride (75 mL). The reaction was stirred at 55° C. overnight. The reaction was concentrated in vacuo. The crude product was taken directly to the next reaction. LC-MS for C₁₁H₁₂BrNO₂ [M+H⁺]: calculated 270.0, found 270.2.
Step C

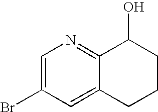

To a solution of crude product from Step B (approx 70 mmol) in methanol (300 mL) was added potassium carbonate (37 g, 268 mmol). The reaction was stirred at ambient temperature for 2 hours. The reaction was concentrated in vacuo. The residue was partitioned between water (500 mL) and ethyl acetate (750 mL). The aqueous layer was extracted with ethyl acetate (750 mL). The combined organic layers were washed with water (200 mL) and brine (200 mL), dried with sodium sulfate, and concentrated in vacuo. The residue was purified by MPLC (Biotage 40+M silica column, 20% to 60% EtOAc/CH₂Cl₂). The combined product fractions were concentrated in vacuo to yield pure product. LC-MS for C₉H₁₀BrNO [M+H⁺]: calculated 228.0, found 228.0.
Step D

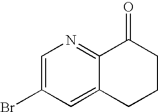

To a solution of product from Step C (1.39 g, 6.08 mmol) in dichloromethane (10 mL) was added Dess Martin periodane (15 wt % sol in CH₂Cl₂, 30 g, 10.6 mmol). The reaction was stirred at ambient temperature for 2.5 hours. The reaction was concentrated in vacuo. The residue was purified by MPLC (Biotage 40+M silica column, 0% to 20% EtOAc/CH₂Cl₂). The combined product fractions were concentrated in vacuo to yield Intermediate 6-6. LC-MS for C₉H₈BrNO [M+H⁺]: calculated 226.0, found 226.1.

### Intermediate 6-7

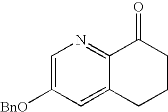

Prepared from 3-benzyloxy-5,6,7,8-tetrahydroquinoline according to the procedure for Intermediate 6-6. LC-MS for C₁₆H₁₆NO₂ [M+H⁺]: calculated 254.1, found 254.1.

### Example 4

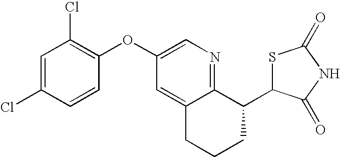 Step A

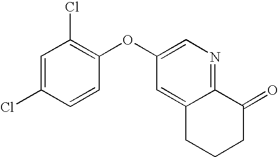

To a mixture of Intermediate 6-6 (300 mg, 1.33 mmol), cesium carbonate (626 mg, 1.92 mmol), and 2,4-dichlorophenol (325 mg, 2.0 mmol) was added N,N-dimethylformamide (6 mL). The reaction vessel was evacuated and filled with nitrogen. The reaction was stirred at 140° C. for 4 hours. The reaction was cooled and was then poured into water (100 mL). The product was extracted with ethyl acetate (2×250 mL). The combined organic layers were washed with water (2×100 mL) and brine (100 mL), dried with sodium sulfate, and concentrated in vacuo. The residue was purified by MPLC (Biotage 0.25+M silica column, 0% to 20% EtOAc/CH₂Cl₂). The combined product fractions were concentrated in vacuo to yield pure product. LC-MS for C₁₅H₁₁Cl₂NO [M+H⁺]: calculated 308.0, found 308.1.
Step B

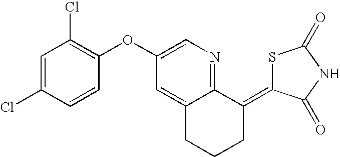

To product from Step A (146.7 mg, 0.476 mmol) was added 2,4-thiazolidinedione (55.8 mg, 0.476 mmol) and sodium acetate (78.1 mg, 0.952 mmol). The reagents were mixed to achieve a homogenous powder. The reaction was placed under vacuum (100 mm Hg) and heated at 160° C. for 1.5 hours. The residue was partitioned between water (30 mL) and ethyl acetate. The product was extracted with ethyl acetate (2×250 mL). The combined organic layers were washed with water (2×100 mL) and brine (100 mL), dried with sodium sulfate, and concentrated in vacuo. The residue was purified by MPLC (Biotage 25+M silica column, 0% to 20% EtOAc/CH₂Cl₂). The combined product fractions were concentrated in vacuo to yield pure product. LC-MS for C₁₈H₁₂C₁₂N₂O₃S [M+H⁺]: calculated 407.0, found 407.2.
Step C

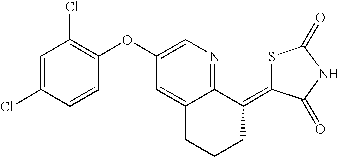

To product from Step B (100.0 mg, 0.246 mmol) was added pyridine (0.5 mL), tetrahydrofuran (0.5 mL), and lithium borohydride solution (2 M in THF, 1.1 mL, 2.2 mmol). The reaction was evacuated, flushed with nitrogen, and heated at 90° C. for 6 hours. The reaction was cooled and then quenched with methanol. The reaction was concentrated in vacuo. The residue was purified by reverse phase HPLC (YMC-Pack Pro C18 5 micron, 40% to 100% CH₃CN/H₂O/0.1% TFA). The product fractions were concentrated in vacuo. The crude product was then purified by prep TLC (1000 microns, silica, 15% EtOAc/CH₂Cl₂) to yield pure product as a mixture of diastereomers. This mixture was further purified by chiral HPLC (ChiralPac OD, 25% EtOH/Heptane isocratic) and yield pure product (more polar major peak, 8 mg, 8%) as single enantiomer. LC-MS for C₁₈H₁₄Cl₂N₂O₃S [M+H⁺]: calculated 409.0, found 409.0.

### Example 5

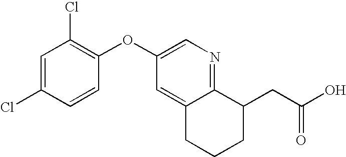 Step A

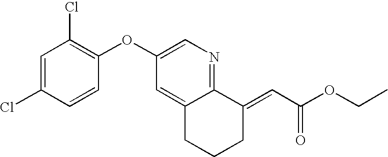

To a mixture of product from Step A in Example 4 (70 mg, 0.227 mmol) and sodium hydride (60% in oil, 27.3 mg, 0.682 mmol) was added tetrahydrofuran (0.5 mL) and triethyl phosphonoacetate (137 μL, 0.682 mmol). The reaction vessel was evacuated and filled with nitrogen. The reaction was stirred overnight at ambient temperature. The reaction was concentrated in vacuo. The residue was purified by prep TLC (1000 microns, silica, 4% EtOAc/CH₂Cl₂) to yield pure product. LC-MS for C₁₉H₁₇Cl₂NO₃[M+H⁺]: calculated 378.1, found 378.1.
Step B

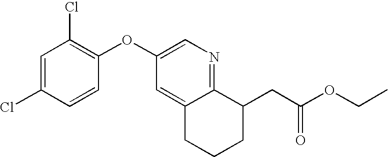

To product from Step B (45 mg, 0.12 mmol) in anhydrous ethanol (10 mL) was added 10% palladium on carbon (45 mg). The reaction vessel was evacuated and filled with hydrogen (1 atm). The reaction was stirred at ambient temperature for 30 min. The catalyst was then removed by vacuum filtration. The filtrate was concentrated in vacuo. The residue was purified by reverse phase HPLC (YMC-Pack Pro C18 5 micron, 40% to 100% CH₃CN/H₂O/0.1% TFA). The combined pure fractions were lyophilized overnight to obtain pure product. LC-MS for C₁₉H₁₉Cl₂NO₃ [M+H⁺]: calculated 380.1, found 380.3.
Step C

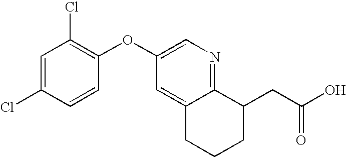

To a mixture of product from Step B (20 mg, 0.053 mmol) and lithium hydroxide monohydrate (15 mg, 0.36 mmol) was added methanol/tetrahydrofuran/water (2:2:1 mixture, 8 mL). The reaction was stirred overnight under ambient conditions. The reaction was concentrated in vacuo. The residue was purified by reverse phase HPLC (YMC-Pack Pro C18 5 micron, 20% to 80% CH₃CN/H₂O/0.1% TFA). The combined pure fractions were lyophilized overnight to obtain pure product. LC-MS for C₁₇H₁₅C₁₂NO₃ [M+H⁺]: calculated 352.1, found 352.1.

### Example 6

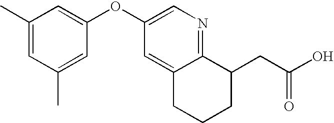 Step A

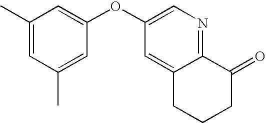

Prepared from Intermediate 6-6 and 3,5-dimethyl-phenol according to the procedure for the product in Step A in Example 4. LC-MS for C₁₇H₁₈NO₂ [M+H⁺]: calculated 268.1, found 268.2.
Step B

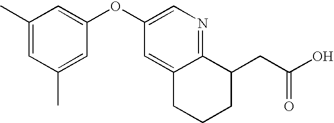

Prepared from the product of Step A according to the procedure for Example 5. LC-MS for C₁₉H₂₂NO₃ [M+H⁺]: calculated 312.1, found 312.2.

### Example 7

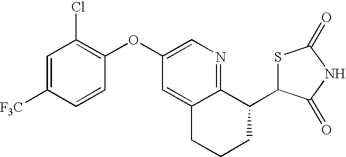 Step A

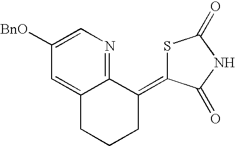

To Intermediate 6-6 (1.13 g, 4.48 mmol) was added 2,4-thiazolidinedione (551 mg, 4.7 mmol) and sodium acetate (385.5 mg, 4.7 mmol). The reagents were mixed to achieve a homogenous powder. The reaction was heated at 160° C. overnight under a nitrogen atmosphere. The residue was partitioned between water (200 mL) and ethyl acetate (200 mL). The aqueous layer was washed with ethyl acetate (200 mL). The combined organic fractions were washed with brine (100 mL), dried with sodium sulfate, and then concentrated in vacuo. The crude solid was triturated with dichloromethane and ethyl acetate to yield crude product. LC-MS for C₁₉H₁₆N₂O₃S [M+H⁺]: calculated 353.1, found 353.3.
Step B

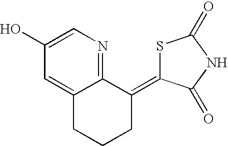

To crude product from Step A (400 mg, 1.135 mmol) in anhydrous ethanol (30 mL) and tetrahydrofuran (35 mL) was added 10% palladium on carbon (400 mg) and hydrochloric acid (2 M in THF, 1.14 mL, 2.28 mmol). The reaction vessel was evacuated and filled with hydrogen (1 atm). The reaction was stirred at ambient temperature for 3 hours. The reaction was degassed and the catalyst was removed by vacuum filtration through celite. The filtrate was concentrated in vacuo to yield pure product. LC-MS for C₁₂H₁₀N₂O₃S [M+H⁺]: calculated 263.1, found 263.2.
Step C

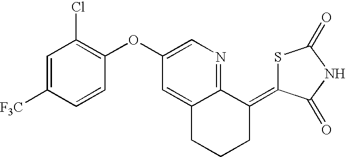

To the product from Step B (301 mg, 0.98) was added 3-chloro-4-fluorobenzotrifluoride (214 mg, 1.08 mmol), cesium carbonate (1.30 g, 4.0 mmol), and N,N-dimethylformamide (10 mL). The reaction was heated at 140° C. for 4 hours under a nitrogen atmosphere. The excess base was removed by syringe filtration, and to the filtrate was added acetic acid (0.5 mL). The filtrate was purified by reverse phase HPLC (YMC-Pack Pro C18 5 micron, 40% to 100% CH₃CN/H₂O/0.1% TFA). The combined product fractions were lyophilized to yield pure product. LC-MS for C₁₉H₁₂ClF₃N₂O₃S [M+H⁺]: calculated 441.0, found 441.3.
Step D

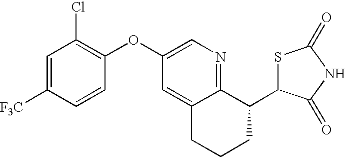

To the product from Step C (270.0 mg, 0.612 mmol) was added pyridine (5 mL), tetrahydrofuran (5 mL), and lithium borohydride solution (2 M in THF, 3.1 mL, 6.2 mmol). The reaction was evacuated, flushed with nitrogen, and heated at 80° C. overnight. The reaction was cooled and then quenched with methanol. The reaction was concentrated in vacuo. The residue was purified by reverse phase HPLC (YMC-Pack Pro C18 5 micron, 40% to 100% CH₃CN/H₂O/0.1% TFA). The product fractions were lyophilized to yield pure product as mixture of diastereomers. This mixture was purified by chiral HPLC (ChiralPac AS, 30% IPA/Heptane isocratic). The less polar major peak was collected to yield the desired product as single enantiomer. LC-MS for C₁₉H₁₄ClF₃N₂O₃S [M+H⁺]: calculated 443.0, found 443.0.

### Intermediate 7

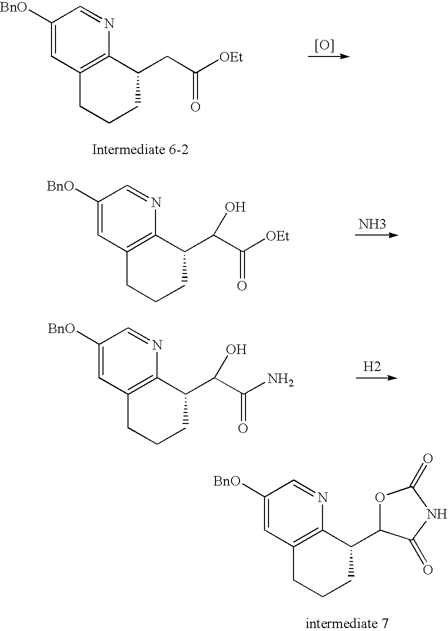 Step A

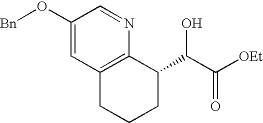

To a solution of intermediate 6-2 (110 mg, 0.336 mmol) in THF was added NaHMDS at −78° C. After 30 minutes, Davis' oxidant (128 mg, 0.49 mmol, prepared as reported in J. of Am. Chem. Soc., 1980, 102, 2004) was added in one portion and the reaction was allowed to warm to room temperature over one hour and then quenched with saturated NaHCO₃ (100 mL). The mixture was extracted with EtOAc (3×80 mL). The organic layers were combined, washed with Brine (1×50 mL), dried over anhydrous Na₂SO₄, filtered and concentrated in vacuo. The residue was purified by reverse phase HPLC (YMC-Pack Pro C18 5 micron, 10% to 100% CH₃CN/H₂O/0.1% TFA) to give the desired product as a white solid. LC-MS for C20H24NO4 [M+H⁺]: calculated 342, found 342.
Step B

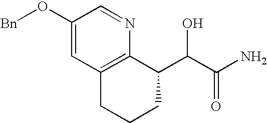

The hydroxyl ester from Step A (70 mg) was mixed with 7 N ammonia-methanol (10 mL) and heated at 55° C. for 5 days in a sealed tube. The reaction was then concentrated in vacuo and the residue was purified by reverse phase HPLC (YMC-Pack Pro C18 5 micron, 10% to 100% CH₃CN/H₂O/0.1% TFA) to give the desired product as a white solid. LC-MS for C₁₈H₂₁N₂O₃ [M+H⁺]: calculated 312, found 312.
Step C

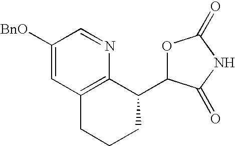

The hydroxy amide (280 mg, 0.9 mmol) from step B and diethyl carbonate (747 mg, 6.335 mmol) was mixed with sodium methoxide (345 mg, 6.335 mmol) and ethanol (10 mL). The mixture was refluxed for 3 hours and then evaporated. The residue was purified by reverse phase HPLC (YMC-Pack Pro C18 5 micron, 10% to 100% CH₃CN/H₂O/0.1% TFA) to give the desired product as a white solid. LC-MS calc. for C19H₁₈N₂O4: 338; Found: 339 (M+H).
Step D

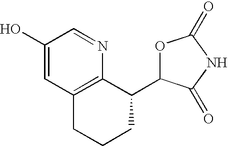

To a solution of the product from step C (180 mg) in ethanol (15 mL) was added 10% Pd/C (200 mg) followed by 4 N HCl/dioxane (2 mL). The reaction was hydrogenated at 50 psi in a par-shaker for 2 hours. The mixture was then filtered through a pad of celite and the filtrate was concentrated in vacuo to give a white solid as HCl salt. LC-MS: calc. for C12H12IN₂O4 248 Found: 249 (M+H).

### Example 8

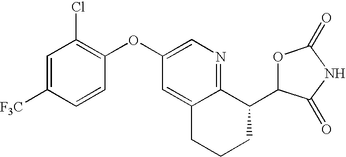

To intermediate 7 (50 mg, 0.176 mmol) was added 3-chloro-4-fluorobenzotrifluoride (70 mg, 0.352 mmol), cesium carbonate (172 mg, 0.528 mmol), and N,N-dimethylformamide (1 mL). The reaction was heated at 110° C. for 23 hours under a nitrogen atmosphere. The excess base was removed by syringe filtration, and to the filtrate was added acetic acid (0.5 mL). The filtrate was purified by reverse phase HPLC (YMC-Pack Pro C18 5 micron, 40% to 100% CH₃CN/H₂O/0.1% TFA). The combined product fractions were lyophilized to yield pure product as TFA salt. LC-MS for C₁₉H₁₄ClF₃N₂O₄ [M+H⁺]: calculated 426, found 427 [M+H].

### Example 9

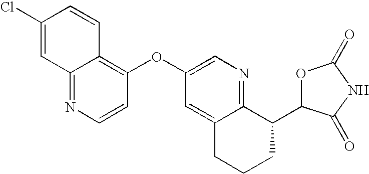

Prepared from intermediate 7 and 4,7-dichloroquinoline according to the procedure for Example 8. LC-MS for C21H16ClN3O4: calculated 409, found 410 [M+H⁺].

### Example 10

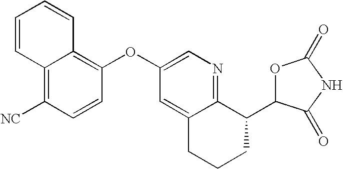

Prepared from intermediate 7 and 1-cyano-4-fluoronaphthalene according to the procedure for Example 8. LC-MS for C23H17N3O4: calculated 399, found 400 [M+H⁺].

### Example 11

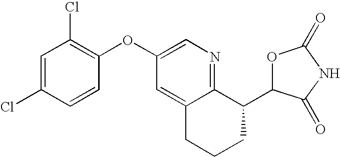

Prepared from intermediate 7 and 1,3-dichloro-4-fluorobenzene according to the procedure for Example 8. LC-MS for C18H14Cl2N2O4: calculated 392, found 393 [M+H⁺].

### Example 12

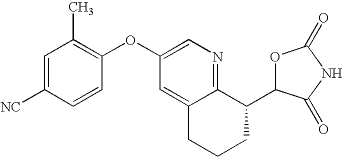

Prepared from intermediate 7 and 4-fluoro-3-methylbenzonitrile according to the procedure for Example 8. LC-MS for C20H17N3O4: calculated 363, found 364 [M+H⁺].

### Example 13

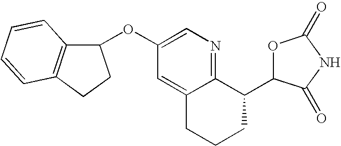

Prepared from intermediate 7 and 1-chloroindane according to the procedure for Example 8. LC-MS for C21H20N2O4: calculated 364, found 365 [M+H⁺].

1. A compound of formula I: 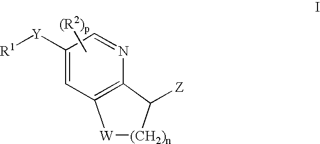
or a pharmaceutically acceptable salt thereof, wherein:
Z is selected from the group consisting of —CR³R⁴CO₂R⁵, —OCR³R⁴CO₂R⁵, —N(R⁶)CR³R⁴CO₂R⁵, —SCR³R⁴CO₂R⁵, tetrazole; and the heterocyclic ring II: 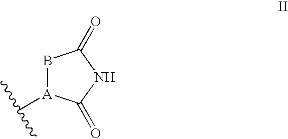
wherein A is —N— or —CR⁹—;
B is selected from S, —NR⁶—, —CH₂—, and O;
Y is selected from the group consisting of O, S, —C(═O)—, and —NR⁶—;
W is selected from O, S, —CH₂—, —CF₂—, and —NR⁶—;
R¹ is a cyclic substituent group selected from the group consisting of phenyl, naphthyl, C₃-C₆ cycloalkyl, indanyl, indenyl, tetrahydronaphthyl, 2,3-dihydrobenzofuranyl, benzopyranyl, 1,4-benzodioxanyl, pyridinyl, pyrazinyl, pyrimidinyl, furyl, pyrrolyl, thiophenyl, imidazolyl, oxazolyl, thiazolyl, isoquinolyl, isoxazolyl, isothiazolyl, pyrazolyl, oxadiazolyl, thiadiazolyl, triazolyl, tetrazolyl, triazinyl, thienyl, pyridazinyl, pyrazinyl, benzisoxazolyl, benzoxazolyl, benzothiazolyl, benzimidazolyl, benzofuranyl, benzothiophenyl (including S-oxide and dioxide), furo(2,3-b)pyridyl, quinolyl, indolyl, quinazolinyl, and dibenzozofuranyl, wherein said R¹ is optionally substituted with 1-3 substituents independently selected from halogen, —OH, —CN, —NO₂, —NR⁷R⁸, C₁-C₃ alkyl, —OC₁-C₅ alkyl, —C(═O)C₁-C₃ alkyl, and —S(O)_{q}C₁-C₃ alkyl, wherein C₁-C₃ alkyl and the alkyl groups of —OC₁-C₅ alkyl, —C(═O)C₁-C₃ alkyl, and —S(O)_{q}C₁-C₃ alkyl are optionally substituted with 1-3 halogens;
R² is selected from the group consisting of halogen, —OH, —CN, —NO₂, —NR⁷R⁸, C₁-C₃ alkyl, and —OC₁-C₃ alkyl, wherein C₁-C₃ alkyl and the alkyl group of —OC₁-C₃ alkyl are optionally substituted with 1-3 halogens;
R³ and R⁴ are each independently selected from the group consisting of H and C₁-C₃ alkyl, which is optionally substituted with 1-3 F;
R⁵ is selected from the group consisting of H and C₁-C₆ alkyl, which is optionally substituted with 1-3 F;
R⁶, R⁷, and R⁸ are each independently selected from the group consisting of H and C₁-C₃ alkyl;
R⁹ is selected from the group consisting of H, C₁-C₃ alkyl, and CF₃;
n is an integer from 1-3;
p is 0, 1, or 2; and
q is 0, 1, or 2.

2. The compound according to claim 1, or a pharmaceutically acceptable salt thereof, wherein R¹ is selected from the group consisting of phenyl, 2-pyridinyl, quinolyl, indanyl, and naphthyl, wherein R¹ is optionally substituted with 1-3 substituents independently selected from halogen, —OH, —CN, —NO₂, —NR⁷R⁸, C₁-C₃ alkyl, —OC₁-C₅ alkyl, —C(═O)C₁-C₃ alkyl, and —S(O)_{q}C₁-C₃ alkyl, wherein C₁-C₃ alkyl and the alkyl groups of —OC₁-C₅ alkyl, —C(═O)C₁-C₃ alkyl, and —S(O)_{q}C₁-C₃ alkyl are optionally substituted with 1-3 halogens;
R³, R⁴, R⁵, and R⁶ are H;
R⁷ and R⁸ are independently selected from H and CH₃;
R⁹ is selected from H and C₁-C₃ alkyl; and
p is 0.

3. The compound according to claim 2, or a pharmaceutically acceptable salt thereof, wherein R⁷ and R⁸ are H; and R⁹ is selected from H and CH₃.

4. The compound according to claim 3, or a pharmaceutically acceptable salt thereof, wherein R⁹ is H.

5. The compound according to claim 2, or a pharmaceutically acceptable salt thereof, wherein R¹ is substituted with 1-3 groups independently selected from F, Cl, Br, CH₃, CF₃, —OCH₃, —OCF₃, —CN, —NO₂, and —OH.

6. The compound according to claim 1, or a pharmaceutically acceptable salt thereof, wherein Z is selected from —CH₂CO₂H and the heterocyclic ring IIa: 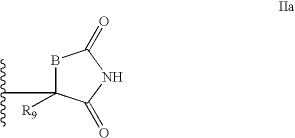
wherein R⁹ is selected from H and C₁-C₃ alkyl, and B is selected from S, O, and —NH—.

7. The compound according to claim 1, or a pharmaceutically acceptable salt thereof, wherein Y is O.

8. The compound according to claim 1, or a pharmaceutically acceptable salt thereof, wherein W is —CH₂—; and n is 1 or 2.

9. The compound of claim 1 having Formula Ia: 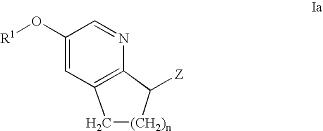
or a pharmaceutically acceptable salt thereof, wherein:
Z is selected from the group consisting of —CH₂CO₂R⁵ and the heterocyclic ring IIa: 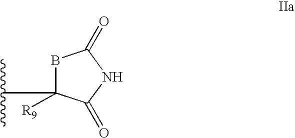
wherein B is selected from S, O, and —NH—;
R¹ is phenyl, 2-pyridinyl, indanyl, quinolyl, or naphthyl, wherein R¹ is optionally substituted with 1-3 substituents independently selected from F, Cl, Br, CH₃, CF₃, —OCH₃, —OCF₃, —CN, —NO₂, and —OH;
R⁵ is selected from the group consisting of H and C₁-C₆ alkyl, which is optionally substituted with 1-3 F;
R⁹ is selected from H and C₁-C₃ alkyl; and
n is 1 or 2.

10. The compound of claim 9, or a pharmaceutically acceptable salt thereof, wherein R⁵ and R⁹ are H; and
B is S or O.

11. The compound of claim 10, or a pharmaceutically acceptable salt thereof, wherein R¹ is phenyl or 2-pyridinyl, wherein R¹ is substituted with 2 substituents independently selected from F, Cl, CH₃, and CF₃.

12. The compound of claim 1 having Formula Ib: 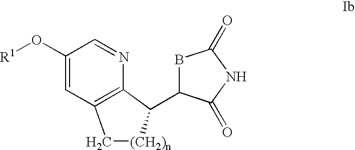
or a pharmaceutically acceptable salt thereof, wherein:
R¹ is phenyl, 2-pyridinyl, indanyl, quinolyl, or naphthyl, wherein R¹ is optionally substituted with 1-3 substituents independently selected from F, Cl, Br, CH₃, CF₃, —OCH₃, —OCF₃, —CN, —NO₂, and —OH;
B is selected from S, O, and —NH—; and
n is 1 or 2.

13. The compound of claim 12, or a pharmaceutically acceptable salt thereof, wherein B is S or O; and R¹ is phenyl or 2-pyridinyl

14. The compound of claim 10, or a pharmaceutically acceptable salt thereof, which is selected from the group of compounds consisting of:

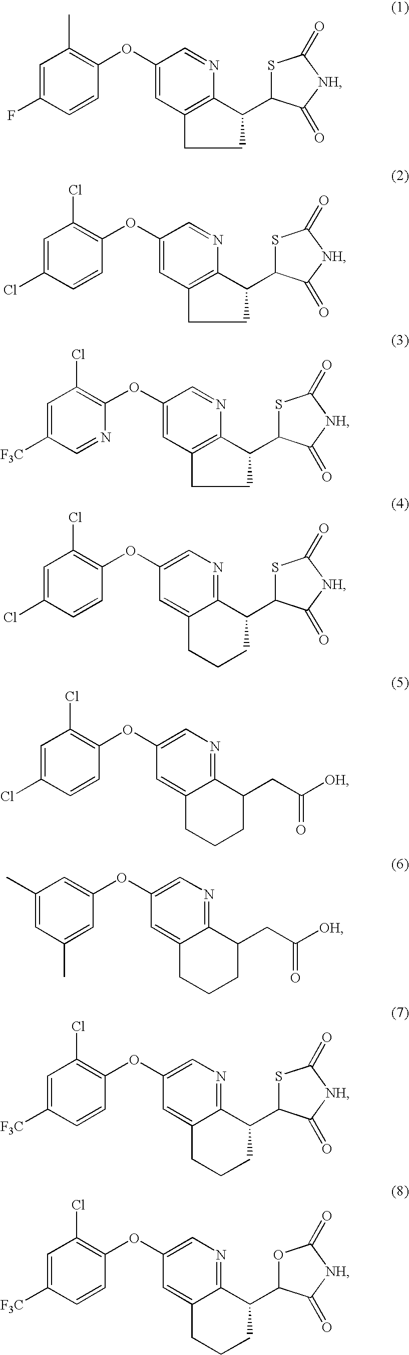

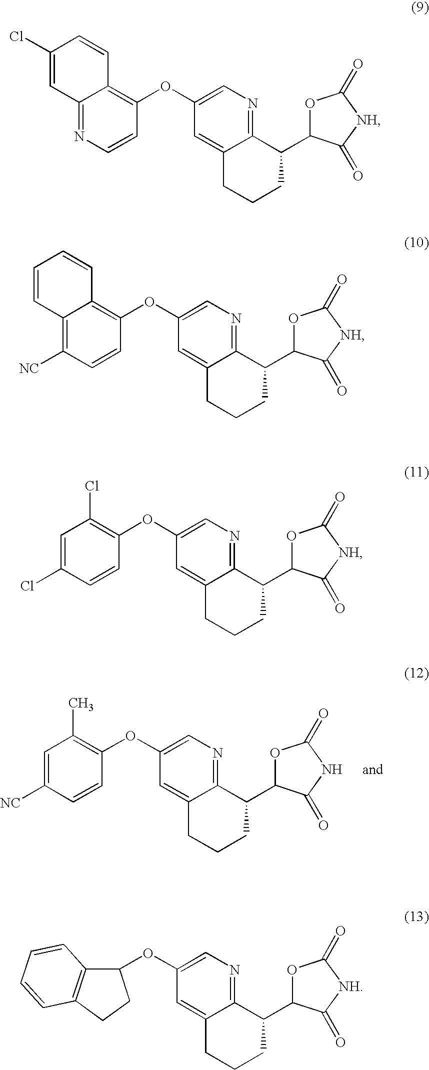

15. A pharmaceutical composition comprising a compound of claim 1, or a pharmaceutically acceptable salt thereof, and a pharmaceutically acceptable carrier.

16. (canceled)

17. A pharmaceutical composition comprising
(1) a compound of claim 1 or a pharmaceutically acceptable salt thereof;
(2) one or more compounds selected from the group consisting of:
(a) PPAR gamma agonists and partial agonists;
(b) biguanides;
(c) protein tyrosine phosphatase-1B (PTP-1B) inhibitors;
(d) dipeptidyl peptidase IV (DP-IV) inhibitors;
(e) insulin or an insulin mimetic;
(f) sulfonylureas;
(g) α-glucosidase inhibitors;
(h) agents which improve a patient's lipid profile, said agents being selected from the group consisting of (i) HMG-CoA reductase inhibitors, (ii) bile acid sequestrants, (iii) nicotinyl alcohol, nicotinic acid or a salt thereof, (iv) PPARα agonists, (v) cholesterol absorption inhibitors, (h) acyl CoA:cholesterol acyltransferase (ACAT) inhibitors, (i) CETP inhibitors, and (j) phenolic anti-oxidants;
(i) PPARαγdual agonists,
(j) PPARδ agonists,
(k) antiobesity compounds,
(l) ileal bile acid transporter inhibitors;
(m) anti-inflammatory agents;
(n) glucagon receptor antagonists;
(o) GLP-1;
(p) GIP-1;
(q) GLP-1 analogs; and
(r) HSD-1 inhibitors; and
(3) a pharmaceutically acceptable carrier.

18. A method of treating type 2 diabetes comprising the administration of a therapeutically effective amount of the compound of claim 1, or a pharmaceutically acceptable salt thereof, to a patient in need of treatment.
